(19) United States
(12) Patent Application Publication
(10) Pub. No.: US 2003/0092100 A1
(43) Pub. Date: May 15, 2003

(54) Tn7 transposition method for producing mutant genes

(76) Inventors: Sze-Chung Clive Lo, Durham, NC (US); Lisbeth Hamer, Durham, NC (US); Jeffrey Shuster, Chapel Hill, NC (US)

Correspondence Address: PARADIGM GENETICS, INC, 108 ALEXANDER DRIVE, P O BOX 14528, RTP, NC, 27709-4528, US

(21) Appl. No.: 10/010,288

(22) Filed: Nov. 13, 2001

(51) Int. Cl. C12Q 1/68; C12N 1/16; C12N 15/74; C12P 21/02

(52) U.S. Cl. 435/69.1; 435/254.2; 435/483; 435/320.1; 435/6; 435/473

(57) ABSTRACT

Surprisingly, the present inventors have discovered that Tn7, a prokaryotic transposon, carries mRNA 3′ end formation site information unique to eukaryotic genes. In vivo gene disruption by Sif, a Tn7-based transposon cassette, in eukaryotic cells can result in pre-mature termination of transcription, yet the resulting mRNA does not appear to rapidly decay as might be expected. These truncated messages are chimeric and polyadenylated. Sif transposons, therefore, can be used for in vitro transposition of selected genes and the resulting construct for in vivo gene replacement in fungi and other eukaryotes. Thus, the present invention provides a method for altering the expression of genes of interest in filamentous fungi and eukaryotes. The resulting mutant genes can be isolated and are useful for identification of functional domains in genes/proteins by methods including, but not limited to, yeast complementation assays or in vitro assays. The methods of the invention are also useful for the generation of leaky mutations to study lethal genes.

### FIELD OF THE INVENTION

The invention relates generally to molecular biology in eukaryotes. In particular, the invention relates to methods for altering the expression of genes in filamentous fungi and other eukaryotes and identifying different functional domains of said genes and their gene products.

### BACKGROUND OF THE INVENTION

Control of gene expression is achieved through a number of diverse mechanisms. Recently, mRNA degradation has been shown to be a powerful and frequently used mode of post-transcriptional regulation in cells (Ruiz-Echevarria et al. (1996): Trends Biochem Sci 21: 433-8 (PMID: 8987399)). Transcripts lacking a translational stop codon or containing premature stop codons are liable to translate to defective proteins with potentially deleterious effects. It is in such instances that mRNA degradation, or more appropriately the nonsense-mediated mRNA decay pathway, is seen to be responsible for the elimination of the aberrant transcripts. Ribosomes which encounter such defective transcripts are thought to activate a scanning complex that recognizes certain 3′ downstream elements (DSEs), resulting in the formation of defective ribonucleoprotein (RNP) structure, leaving the mRNA susceptible to decapping by exoribonucleases such as Xrn1 (Ruiz-Echevarria et al. (1996): Trends Biochem Sci 21: 433-8 (PMID: 8987399)).

mRNAs that contain premature stop codons (nonsense mutations)—presumably preventing recognition of the now more distant DSEs—are unstable in all eukaryotes. Mutation can also destabilize mRNA by the removal or impairment of stabilizing structures, such as some 3′ stem-loops (Kuhn et al. (2001) Mol Cell Biol 21: 731-42 (PMID: 11154261)). Regardless of their “normal” decay rates, mRNAs transcribed from genes that harbor nonsense mutations (dubbed nonsense-containing mRNAs) are degraded very rapidly. Such “nonsense-mediated mRNA decay” is ubiquitous, i.e., it has been observed in all organisms tested, and leads to as much as ten- to one-hundred-fold reduction in the abundance of specific mRNAs. The combination of severely reduced mRNA abundance and prematurely terminated translation causes reductions in the overall level of expression of specific genes that are as drastic as the consequences of gene deletion. Premature termination of transcripts in a manner that hinders or prevents nonsense-mediated decay might allow for the in vivo study of truncated gene products—a desirable tool permitting the dissection of gene function.

Transposons can be used to truncate target genes, and transposable elements often cause a variety of effects on the expression of target genes. A number of eukaryotic transposons are known to carry polyadenylation signals that cause premature termination of target gene transcription, e.g. gypsy (Drosophila, Hoover et al. (1993) Genetics 135: 507-26 (PMID: 8244011)), Mu1 (maize, Ortiz and Strommer (1990) Mol Cell Biol 10: 2090-95 (PMID: 2157968)), Tnt1 (tobacco, Pouteau et al. (1991) Mol Gen Genet 228: 233-9 (PMID: 1715973)), Fot1 (Fusarium oxysproum, Deschamps et al. (1999) Mol Microbiol 31: 1373-83 (PMID: 10200958)). As a result, truncated transcripts are often detectable for the genes they interrupt. Transposon insertions that produce truncated mRNAs and prevent rapid mRNA degradation would be an excellent tool to facilitate the in vivo study of truncated gene products.

### SUMMARY OF THE INVENTION

Surprisingly, the present inventors have discovered that Tn7, a prokaryotic transposon, carries mRNA 3′ end formation site information unique to eukaryotic genes. The inventors developed Sif, a bacterial Tn-7-based transposon cassette, for use in generating genome-wide single gene mutants in M. grisea and M. graminicola (TAG-KO technology, see WO 00/55346, PCT/US00/07317, U.S. Ser. No. 09/658,859). Sif allows in vitro transposition of selected genes and the use of the resulting construct for in vivo gene replacement in fungi. The inventors have further discovered that Sif, like some eukaryotic transposons, can terminate gene transcription in an eukaryotic manner. In vivo gene disruption by Sif in eukaryotic cells can result in pre-mature termination of transcription, yet the resulting mRNA does not appear to rapidly decay as might be expected. Truncated transcripts were detected for the target gene cystathione beta synthase (CBS) (FIGS. 2 and 3), and they were polyadenylated (SEQ ID NO: 1, 2, and 3). It is a novel discovery that Tn7, a prokaryotic transposon carries 3′-end formation site information unique to eukaryotic genes.

Sif transposons, therefore, can be used to alter the expression of genes of interest in filamentous fungi, and to generate libraries of mutants, such as truncation mutants, as opposed to the more limited, less random permutations of deletions that can be made with restriction enzymes. The inventors can isolate truncated messages from such libraries of mutants and identify functional domains in genes/proteins by using assays including, but not limited to, yeast complementation assays or in vitro binding assays. Additionally, leaky mutations can be generated using this system to study lethal genes.

Thus, the present invention provides A method for preparing a functionally distinct mutant form of a gene and/or a mutant gene product comprising: inserting at least one Tn7 transposon in vitro into a gene to produce a mutant form of said gene; and assaying said mutant gene and/or its mutant gene product for a change in activity or function compared to said activity or function for the gene and/or its gene product; wherein a change indicates the mutant form and/or its mutant gene product is functionally distinct from said gene and/or its gene product. The present invention also provides a method for preparing a functionally distinct mutant form of a gene and/or a mutant gene product comprising: inserting at least one Tn7 transposon in vitro into a gene to produce a mutant form of said gene; transforming at least one host with said mutant form to give a transformant; and assaying: said mutant gene and/or its mutant gene product for a change in activity or function compared to said activity or function for the gene and/or its gene product, and/or said transformant for a change in phenotype; wherein a change detected in either or both indicates the mutant form and/or its mutant gene product is a functionally distinct mutant form of said gene and/or its gene product.

One advantage of the present invention is that the mutant genes produced by this method express stable mRNAs, thereby facilitating the study of gene function. This method also allows a library of mutants to be produced, and for the position of the mutations produced to be more random than by other methods.

### BRIEF DESCRIPTION OF THE FIGURES

FIG. 1 The CBS gene and TAG-KO mutation alleles in M. grisea. The CBS gene in M. grisea contains two exons (boxes) separated by an intron. TAG-KO mutants, KO1, KO2, and KO3, harbor transposon insertions (triangles in the gene at the indicated positions). Numbers represent nucleotide positions after the start codon ATG.

FIG. 2 is a digital image showing the effect of CBS gene disruption by transposon insertion on gene expression by Northern analysis in different M. grisea strains. A PCR fragment amplified with primers upstream of all the transposon sites (FIG. 1) was used as a probe. The difference in sizes of transcripts derived from different strains suggested that transcription was terminated at the transposon insertion site. Two independent transformants for each mutant (KO1, KO2, and KO3) are represented.

FIG. 3 is molecular analysis of CBS expression in different M. grisea strains. A) 3′ ends of CBS cDNAs. KO1, KO2, and KO3 made truncated CBS transcripts with addition of some transposon sequences (grey lines). Numbers indicate positions relative to the stop codon in the wild type (WT) cDNA. Note that new stop codons, TAG and TAA, were identified in the transposon sequence for the CBS gene in KO1 and KO3, respectively. B) Deduced CBS proteins with novel amino acid sequences (letters) as a result of transposn insertions. KO2 is expected to produce the wild type protein.

FIG. 4 is a digital image of a complementation assay in S. cerevisiae of the CBS gene by M. grisea CBS cDNAs. CBS cDNAs (containing a start codon through a poly(A) tail) from M. grisea WT and TAG-KO strains were cloned into pYes2.1 vectors to make expression constructs (pYCBS-WT, pYCBS-KO1, pYCBS-KO2, pYCBS-KO3). S. cerevisiae CBS mutant (6696) was unable to grow in the absence of cysteine on minimal media (MM). Cysteine was not required for the growth of the wild type strain (4471) on MM. The expression constructs were transformed into the strain 6696. pYCBS-WT, pYCBS-KO1, and pYCBS-KO2 rescued the CBS phenotype. The strain transformed with pYCBS-KO3 showed slower growth, suggesting that the truncated gene was partially functional.

### DETAILED DESCRIPTION OF THE INVENTION

Definitions

As used herein, the term “allele” refers to any of the alternative forms of a gene that may occur at a given locus.

The term “altered expression” refers to a change in expression. “Expression” itself, or “gene expression” refers most concisely to the detectable effect of a gene. In part this refers to the process by which a gene's coded information is converted into the structures present and operating in the cell. Expressed genes include those that are transcribed into mRNA and then translated into protein and those that are transcribed into RNA but not translated into protein (e.g., transfer and ribosomal RNAS). While the “detectable effect” may refer to a phenotype, it is more commonly used in molecular biology to refer to these “detectable” products of the process of gene expression—RNA and possibly protein. The observed change in quantitative and/or qualitative assessments of the amount of a detectable gene product (RNA or protein) can thus be viewed as the “effect”. As a result, “over-expression”, “under-expression”, “expression”, “gene expression”, and “level of expression” often refer to the quantity or change in quantity of a gene product (RNA or protein), and/or of the additional gene product that may be generated (e.g., RNA or protein by a gene, or protein by RNA), and/or of the activity of a gene or gene product. Thus, “altered expression” encompasses changes in transcription, post-transcriptional processing, translation, and post-translational modification that lead to changes in the quantity of gene product and/or activity. Expression may be regulated at any of these levels, but is most commonly controlled at the initiation of transcription, depending on cell type, developmental stage, cell cycle stage and/or environmental cues. As the timing of a gene's action and expression are often regulated to only occur, for example, at particular stages of development or of the cell division cycle, a change in gene product stability and/or timing of expression also constitutes “altered expression”.

The term “bDNA” refers to branched DNA.

The term “binding” refers to a noncovalent or a covalent interaction, preferably noncovalent, that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

As used herein “biochemical assay” refers to an assay based on defined characteristics of a biochemical reaction, function, or the reactants, products, or proteins involved.

As used here in “CBS” is synonymous with “cystathione beta synthase”, and refers to the cystathione beta synthase gene and/or gene products of Magnaporthe grisea.

As used herein, the term “cDNA” means complementary deoxyribonucleic acid.

As used herein, the term “complementation assay” refers to an assay to test if two putative alleles, when in the same cell and acting independently, can supply all functions necessary for an apparently wild-type phenotype under the conditions used. Function need not be provided by both alleles. Complementation is therefore a test of function.

As used herein, the term “cosmid” refers to a hybrid vector, used in gene cloning, that includes a cos site (from the lambda bacteriophage). It also contains drug resistance marker genes and other plasmid genes. Cosmids can incorporate larger DNA fragments than either phage or plasmid vectors alone and are especially suitable for cloning large mammalian genes or multigene fragments.

As used herein, the term “DNA” means deoxyribonucleic acid.

As used herein, the term “DSE” refers to a downstream element.

As used herein, the term “enzymatic assay” refers to any assay for detecting an enzymatic function, such as the conversion of substrate to product.

As used herein, the term “functional domain” refers to the region or regions of a DNA, RNA, or amino acid sequence required for or contributing to a particular activity or function. Functional domains for differing activities or functions may overlap or be identical.

As used herein the term “functionally distinct” means having a detectable difference in function or activity.

“Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia, perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. They can either exist as single cells or make up a multicellular body called a mycelium, which consists of filaments known as hyphae. Most fungal cells are multinucleate and have cell walls, composed chiefly of chitin. Fungi exist primarily in damp situations on land and, because of the absence of chlorophyll and thus the inability to manufacture their own food by photosynthesis, are either parasites on other organisms or saprotrophs feeding on dead organic matter. The principal criteria used in classification are the nature of the spores produced and the presence or absence of cross walls within the hyphae. Fungi are distributed worldwide in terrestrial, freshwater, and marine habitats. Some live in the soil. Many parasitic fungi cause disease in animals and man or in plants, while some saprotrophs are destructive to timber, textiles, and other materials. Some fungi form associations with other organisms, most notably with algae to form lichens.

“Gene” or “genetic locus” refers to a unit of heredity. Each gene is composed of a linear chain of deoxyribonucleotides, which can be referred to by the sequence of nucleotides forming the chain. Thus, “gene sequence” or “sequence” is used to indicate both the ordered listing of the nucleotides which form the chain, and the chain itself, which has that sequence of nucleotides, or DNA or RNA products the gene sequence acts as a template. (“Sequence” is used in the same way in referring to RNA chains, linear chains made of ribonucleotides.) The gene may include regulatory and control sequences, as well as sequences which can be transcribed into an RNA molecule, and may contain sequences with unknown function. The majority of the RNA transcription products are messenger RNAs (mRNAs), which include sequences which are translated into polypeptides (coding sequence) and may include sequences which are not translated (untranslated regions or UTRs). It should be recognized that small differences in nucleotide sequence for the same gene can exist between different fungal strains, or even within a particular fungal strain, without altering the identity of the gene.

As used herein, the term “gene disruption” or “knockout” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker. Gene knockouts generally depend on homologous recombination between DNA introduced into the cell and genomic DNA sequences, and allow the study of gene function.

As used herein the term “gene product(s)” refers to the product, either RNA or protein, that results from expression of a gene, or mutant form thereof.

As used herein, the term “genotype” refers to all or part of the genetic constitution of an individual or group.

As used herein, the term “host” refers to an eukaryotic cell or cells, or an organism or organisms.

As used herein, the term “HPLC” means high pressure liquid chromatography.

As used herein, the terms “hph” and “hygromycin” refer to the E. coli hygromycin phosphotransferase gene or gene product, respectively.

As used herein, the term “hygromycin B” refers to an aminoglycosidic antibiotic, used for selection and maintenance of eukaryotic cells containing the E. coli hygromycin resistance gene.

As used herein, the term “interaction trap” refers to the yeast two hybrid system, yeast one hybrid system, yeast three hybrid system, and variations and adaptations thereof such as the reverse two hybrid.

As used herein, the term “in vitro” refers to actions performed outside a living organism.

The term “IPTG” refers to Isopropyl-beta-D-thiogalactoside.

As used herein, the terms “KO1”, “KO2”, and “KO3” refer to specific TAG-KO mutants of the CBS gene or the fungal strains that contain them.

As used herein the term “in vitro binding assay” refers to an assay to assess binding of a gene or gene product to another (not necessarily different) gene product that takes place outside an intact cellular or organismal environment. In assaying for dimerization and other forms of multimerization, the binding detected may be between individual polypeptides of the same type. ELISA assays, co-immunoprecipitation, and other methods are known in the art for assaying in vitro binding.

As used herein, the term “mRNA” means messenger ribonucleic acid.

As used herein, the term “mutant form” refers to a gene or gene product “Mutant form of a gene” is synonymous with “mutant gene” and refers to a gene, which has been altered, either naturally or artificially, changing the nucleotide sequence of the gene, which can result in a change in the amino acid sequence of an encoded polypeptide, and/or mRNA. “Mutant form of a gene product” is synonymous with “mutant gene product” and refers to such altered mRNAs and/or polypeptides. The change in the nucleotide sequence may be of several different types, including, but not limited to, changes of one or more bases for different bases, deletions, and insertions. By contrast, a normal form of a gene is a form commonly found in a natural population of a fungal strain. Commonly a single form of a gene will predominate in natural populations. In general, such a gene is suitable as a normal form of a gene, however, other forms which provide similar functional characteristics may also be used as a normal gene.

As used herein, “nonsense mediated mRNA decay” (NMD) is a conserved process, which leads to the detection of premature termination codons (an in-frame triplet of nucleotides: UAA, UAG or UGA) within a mRNA molecule. Termination (or stop) codons normally signal the end of the stretch of mRNA that is translated into protein, so when one appears early, the result can be a truncated protein that could have deleterious consequences for the host organism. This nonsense mRNA is subsequently targeted for decay thus preventing translation and the consequent production of potentially truncated polypeptides. The decay of this nonsense mRNA occurs at a more rapid rate than if the mRNA were to decay through the default decay pathway. This increased decay rate allows the cell to rapidly remove these mRNAs from the pool of translatable mRNAs.

As used herein, the term “PCR” means polymerase chain reaction.

As used herein, the term “phenotype” refers to the detectable properties of an organism that are produced given a particular genotype.

As used herein, the term “phenotypic assay” refers to any assay for detecting a phenotype, or change in phenotype.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

As used herein “reporter gene construct” refers to a DNA construct or plasmid containing a gene that is used to ‘tag’ another gene or DNA sequence of interest, such as a promoter. Expression of the reporter is easily monitored, and permits the function or whereabouts of the ‘target’ sequence to be tracked. It can help pinpoint which cells contain the tagged gene (for instance, among a population of genetically engineered bacteria) or show the varying degrees of expression of the tagged gene under different cellular conditions. For example, the beta-galactosidase gene (lacZ) is a common reporter gene whose activity can be detected on indicator plates by causing a color change in a dye. One use of reporter genes is to investigate the function of ‘foreign’ promoters in transgenic organisms. The promoter is inserted in a vector ‘upstream’ of the reporter gene, and the vector is allowed to transfect the organism. How well and in what tissues the promoter functions can then be assessed by expression of the reporter, and its expression can be used to test the importance of particular DNA sequences in the promoter or to test the importance of the expression of a transcription factor or a signal transduction pathway on gene expression.

As used herein “reporter transcripts” refers to mRNA transcripts produced from a reporter gene construct.

As used herein, the term “reverse transcriptase-PCR” means reverse transcription-polymerase chain reaction.

The term “reverse two-hybrid” refers to a variation of the yeast two-hybrid system, in which protein-protein interactions increase the transcription of a toxic counterselectable marker, resulting in growth inhibition. The availability of a counterselectable marker significantly extends the possibilities of the two-hybrid system. Most importantly, dissociation of protein-protein interactions can be selected for, and thus protein-protein interactions can be characterized and manipulated genetically. (Vidal et al. (1996) “The reverse two-hybrid system and several of its applications” Yeast Genetics and Molecular Biology, Madison, Wis.).

As used herein, the term “RNA” means ribonucleic acid.

As used herein, the term “RNA polymerase” or “DNA-dependent RNA polymerase” or “RNA polymerase II” refers to an enzyme that catalyses the synthesis of a polymeric RNA molecule from ribose-containing nucleotides using an existing DNA strand as a template. Type I is responsible for the synthesis of ribosomal RNA, type II for messenger RNA synthesis, and type III for transfer RNA synthesis.

As used herein, the term “SDS” means sodium dodecyl sulfate.

As used herein, the term “SDS-PAGE” means sodium dodecyl sulfate-polyacrylimide gel electrophoresis.

As used herein, the term “sequence alteration” refers to a change or predicted change in DNA, RNA, or amino acid sequence.

As used herein, “SIF” refers to the transposon derived from the donor plasmid pSIF (see Example 1).

As used herein, the term “SSCP” refers to single strand conformation polymorphism.

As used herein “TAG-KO” means transposon arrayed gene knockout and refers to the technology described in WO 00/55346, PCT/US00/07317, and U.S. 09/658,859.

As used herein, the term “TATA box” refers to a sequence of nucleotides that serves as the main recognition site for the attachment of RNA polymerase in the promoter region of eukaryotic genes. Located at around 25 nucleotides before the start of transcription, it consists of the seven-base consensus sequence TATAAAA, and is analogous to the Pribnow box in prokaryotic promoters.

As used herein, the term “TLC” means thin layer chromatography.

As used herein, the term “Tn7 transposon” refers to the prokaryotic transposable element Tn7, and modified forms thereof (“Tn7-based”), such as Sif (see Examples; see also, Biery et al. (2000) Nucleic Acids Res 28: 1067-77 (PMID: 10666445); Craig (1995) Cur Top Microbiol Immunol 204: 27-48 (PMID: 8556868)). Tn7 has been most commonly studied in Escherichia coli. “Tn7 transposon” can encompass forms of DNA that do not demonstrably contain Tn7 genes, but which can be made to undergo transposition through use of the Tn7 gene products TnsA and TnsB, which collaborate to form the Tn7 transposase, or modifications thereof. Such DNA is bounded by 5′ and 3′ DNA sequences recognizable by the transposase, which can function as the transposon ends. Examples of Tn7 transposon end sequences may be found in Arciszewska et al. (1991) J Biol Chem 266: 21736-44 (PMID: 1657979), Tang et al. (1995) Gene 162: 41-6 (PMID: 7557414), Tang et al. (1991) Nucleic Acids Res 19: 3395-402 (PMID: 1648205), Biery et al. (2000) Nucleic Acids Res 28: 1067-77 (PMID: 10666445), Craig (1995) Cur Top Microbiol Immunol 204: 27-48 (PMID: 8556868), and other published sources, and should allow transposition given the appropriate Tns proteins. Generally, it is thought that the transposon ends are apposed to the target DNA by TnsA and TnsB. These two Tns proteins are thought to then collaborate to execute the breakage and joining reactions that underlie transposition. The Tns proteins present in the transposition reaction may be from any source, such as endogenous Tns genes or exogenously added protein.

As used herein, the term “transcription” refers to the process in living cells in which the genetic information of DNA is copied into a molecule of messenger RNA (mRNA) as the first step in protein synthesis. Transcription takes place in the cell nucleus or nuclear region. It involves the action of RNA polymerase enzymes in assembling the nucleotides necessary to form a complementary strand of mRNA from the DNA template, and (in eukaryote cells) the subsequent removal of the noncoding sequences from this primary transcript (gene splicing) to form a functional mRNA molecule. Other types of RNA that do not encode protein, e.g. ribosomal RNA and transfer RNA, are also produced by transcription from DNA.

As used herein, the term “transcriptional assay” refers to any assay for detecting a transcriptional function, such as the activation of RNA transcription.

As used herein, the term “transcription factor” refers to a DNA-binding protein that facilitates or represses the transcription of a specific gene or set of genes by RNA polymerase.

As used herein, the term “transcripts” refers to RNA transcripts.

“Transform” or “transformation”, as used herein, refers to the introduction of a polynucleotide (single or double stranded DNA, RNA, or a combination thereof) into a living host by any means. Transformation may be accomplished by a variety of methods, including, but not limited to, agroinfection, electroporation, microinjection, particle bombardment, transfection, transduction, and the like. This process may result in transient or stable (constitutive or regulated) expression of the transformed polynucleotide. By “stably transformed” is meant that the sequence of interest is integrated into a replicon in the cell, such as a chromosome or episome. Transformed cells, tissues and plants encompass not only the end product of a transformation process, but also the progeny thereof which retain the polynucleotide of interest.

As used herein, the term “transformant” refers to an host on which transformation has been performed and to which said DNA and/or RNA has been successfully introduced.

As used herein, the term “transposase” refers to an enzyme that catalyzes transposition.

As used herein, the term “transposition” refers to a complex genetic rearrangement process, reproducible in vitro, involving the movement of a DNA sequence (transposon) from one location and insertion into another, often within or between a genome or genomes, or DNA constructs such as plasmids, bacmids, and cosmids.

As used herein, the term “transposon” (also known as a “transposable element”, “transposable genetic element”, “mobile element”, or “jumping gene”) refers to a mobile DNA element. They can disrupt gene expression or cause deletions and inversions, and hence affect both the genotype and phenotype of the organisms concerned. The mobility of transposable elements has long been used in genetic manipulation, to introduce genes or other information into the genome of certain model systems.

As used herein, the term “wild type” refers to a phenotype or the form of an allele possessed by most members of a population in their natural environment, and/or an allele whose phenotype is wild type. A wild type phenotype may refer to a particular aspect of an organism, a group of aspects, an overall aspect, or a molecular phenotype.

As used herein, the term “WT” refers to a wild type CBS gene or a fungal strain wild type for the CBS gene.

The term “yeast one-hybrid” refers to a variation on the yeast two-hybrid system, used for detecting protein-DNA interactions (Yeast Hybrid Technologies, Li Zhu, editor, BioTechniques, 2000).

The term “yeast three-hybrid” refers to modifications of the yeast two-hybrid system. The third hybrid may be a first one with a RNA or with a small molecule that is a cell permeable chemical inducer of dimerization. Another variation is the alteration of the two-hybrid system vectors or the use of additional vectors to allow expression of additional RNAs or proteins. The numeric value refers to the number of genes/gene products whose interactions are being tested, but not necessarily all constitute fusions or hybrid forms of the genes as the name would seem to imply. This term is also taken to encompass higher ordinal numerations of expression of additional genes/gene products.

The term “yeast two-hybrid” refers to a system first developed in by Fields and Song ((1989) Nature 340: 245-6 (PMID: 2547163)), and since modified others in the art) to identify proteins (and their genes) that interact with known proteins. A leading system for investigation of protein-protein interactions.

Embodiments of the Invention

The present inventors have discovered that Tn7, a prokaryotic transposon, carries mRNA 3′ end formation site information unique to eukaryotic genes. Thus, the inventors are the first to demonstrate that Tn7-based vectors can disrupt eukaryotic genes in a manner such that transcription of the disrupted gene can produce a polyadenylated mRNA encoding a truncated gene product.

Gene disruption is performed in vitro and the integration site selection is random, allowing the generation one or more mutant forms of a gene, gene fragment, or genetic locus. Gene disruption may be performed on a gene, gene fragment, or genetic locus in its native chromosome, or in a vector, including, but not limited to, cosmids, bacmids, and plasmids. The resulting mutant gene, transcripts thereof, or transformants may be isolated or selectively isolated by means known to those in the art including, but not limited to, reverse transcriptase-PCR, PCR, restriction digestion, oligonucleotide synthesis, or phenotypic screening.

Altering the expression of a gene can be useful for the identification of its functional domains and/or the functional domains of gene products encoded thereby, such as proteins and RNAs, by means including, but not limited to, yeast complementation assays, yeast interaction trap assays, biochemical assays, enzymatic assays, transcriptional assays, phenotypic assays, and in vitro binding assays.

Accordingly, the present invention provides a method for preparing a functionally distinct mutant form of a gene and/or a mutant gene product comprising:

a) inserting at least one Tn7 transposon in vitro into a gene to produce a mutant form of said gene; and

b) assaying said mutant gene and/or its mutant gene product for a change in activity or function compared to said activity or function for the gene and/or its gene product;

wherein a change indicates the mutant form and/or its mutant gene product is functionally distinct from said gene and/or its gene product.

Any technique for detecting a change in activity may be used in the methods of the invention. For example, a ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with the gene product of interest or a fragment or variant thereof, the unbound protein is removed and the bound gene product of interest is detected. In a preferred embodiment, a bound gene product of interest is detected using a labeled binding partner, such as a labeled antibody. In a variation of this assay, the gene product of interest is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

A gene or gene product of interest's activity can be detected using in vitro transcription assays in which the synthesis of a reporter mRNA is directly or indirectly detected, if the gene or gene product of interest promotes or inhibits the transcription of target genes. Methods for detection of changes in transcription include Northern blotting, use of the yeast one-hybrid system, CAT assays, luciferase assays, in vitro transcription, and others known to those in the art.

A change in activity may also be detected using in vitro enzymatic assays in which the disappearance of a substrate or the appearance of a product is directly or indirectly detected. Possible detection methods include, but are not limited to, spectrophotometry, mass spectroscopy, thin layer chromatography (TLC) and reverse phase HPLC.

For the in vitro assays, a given DNA, RNA, or protein and derivatives thereof may be purified from a fungus or may be recombinantly produced in and purified from a fungal, bacteria, or eukaryotic cell culture. Preferably proteins are produced using a baculovirus or E. coli expression system. Methods for the purification of proteins and polypeptides are known to those skilled in the art. Likewise, methods for purifying DNA and RNA are known to those skilled in the art.

Also envisioned is the alteration of gene expression in vivo for which the present invention provides a method for preparing a functionally distinct mutant form of a gene and/or a mutant gene product comprising:

a) inserting at least one Tn7 transposon in vitro into a gene to produce a mutant form of said gene;

b) transforming at least one host with said mutant form to give a transformant; and

c) assaying:

1) said mutant gene and/or its mutant gene product for a change in activity or function compared to said activity or function for the gene and/or its gene product, and/or

2) said transformant for a change in phenotype; wherein a change detected in c1) and/or c2) indicates the mutant form and/or its mutant gene product is a functionally distinct mutant form of said gene and/or its gene product.

A change in activity or function may result from a change in expression at the transcriptional or translational level. Transposon insertion into sequences containing regulatory domains in particular, such as promoters or mRNA untranslated regions, could raise (overexpression) or lower (underexpression) expression of gene products, and further understanding functions and causes of such regulation. Altering expression in this manner can also lead to what is known in the art as “leaky” expression—expression of an essential gene for which a viable whole gene knockout isn't obtainable at a minimal level to permit viability and some assessment of gene function. Leaky expression, expression with some remaining function—but not at the level of the wild type allele, can also be used to express toxic gene products at levels at or below those permitting cell or organism viability.

Expression of a gene can be measured by detecting said gene's primary transcript or mRNA, polypeptide, or activity. Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Methods for detecting RNA include, but are not limited to, amplification assays such as quantitative reverse transcriptase-PCR, transcriptional fusions using the gene of interest's promoter or derivatives thereof fused to a reporter gene, and/or hybridization assays such as Northern analysis, dot blots, slot blots, in-situ hybridization, bDNA assays, and microarray assays.

Methods for detecting protein expression include, but are not limited to, immunodetection methods such as Western blots, His Tag and ELISA assays, polyacrylamide gel electrophoresis, mass spectroscopy and enzymatic assays. Also, any reporter gene system may be used to detect protein expression. For detection using gene reporter systems, a polynucleotide encoding a reporter protein is fused in frame with the gene of interest, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2:1104; Prost et al. (1986) Gene 45:107-111), β-galactosidase (Nolan et al. (1988) Proc Natl Acad Sci USA 85:2603-2607), alkaline phosphatase (Berger et al. (1988) Gene 66:10), luciferase (De Wet et al. (1987) Mol Cell Biol 7:725-737), β-glucuronidase (GUS), fluorescent proteins, chromogenic proteins and the like.

Phenotypic assays to detect a change in gene function or activity include, but are not limited to, an assay for rescue or complementation of a phenotype (normally wild type) in a strain harboring a gene disruption or a mutant gene (not necessarily a mutant of the same gene), assays which require a particular phenotype such as growth on a selective media as a read out of functional activity such as the yeast interaction trap, and comparison of a transformant with wild type.

Any technique for detecting or predicting sequence alterations may be used in the methods of the invention to determine changes in gene sequence, preferably those resulting from Tn7-based transposition. Such means may use any technique necessary to isolate or purify a particular RNA, DNA, protein or fragment thereof sufficiently to determine such sequence alterations. Examples of methods known in the art for detecting, determining, or predicting sequence alterations include, but are not limited to, DNA sequencing, protein microsequencing, sequencing of complementary DNA, SSCP analysis, changes in electrophoretic mobility, and PCR sequencing.

### Experimental

Fungal Strains

M. grisea strain Guy11 (Leung et al. (1988) Phytopathology 78: 1227-33) was used as the wild-type fungus. Unless, otherwise indicated, M. grisea was grown in complete medium and minimal medium (MM) (Talbot et al. (1993) Plant Cell 5: 1575-90).

### EXAMPLE 1

### Construction of the Sif transposon

Construction of Sif transposon: Sif was constructed using the GPS3 vector from the GPS-M mutagenesis system from New England Biolabs, Inc. (Beverly, Mass.) as a backbone. This system was based on the bacterial transposon Tn7. The following manipulations were done to GPS3 according to Sambrook et al. (1989) Molecular Cloning, a Laboratory Manual, Cold Spring Harbor Laboratory Press. The kanamycin resistance gene (npt) contained between the Tn7 arms was removed by EcoRV digestion. The bacterial hygromycin B phosphotransferase (hph) gene (Gritz and Davies (1983) Gene 25:179-88) under control of the Aspergillus nidulans trpC promoter and terminator (Mullaney et al. (1985) Mol Gen Genet 199: 37-45) was cloned by a HpaI/EcoRV blunt ligation into the Tn7 arms of the GPS3 vector yielding pSif1. Excision of the ampicillin resistance gene (bla) from pSif1 was achieved by cutting pSifl with XmnI and BglI followed by a T4 DNA polymerase treatment to remove the 3′ overhangs left by the BglI digestion and religation of the plasmid to yield pSif. Top 10F.′ electrocompetent E. coli cells (Invitrogen) are transformed with ligation mixture according to manufacturer's recommendations. Transformants containing the Sif transposon were selected on LB agar (Sambrook et al. (1989) Molecular Cloning, a Laboratory Manual, Cold Spring Harbor Laboratory Press.) containing 50 ug/ml of hygromycin B (Sigma Chem. Co., St. Louis, Mo.).

### EXAMPLE 2

### Insertion of Sif into a Cosmid Containing a Gene of Interest

The cystathione beta synthase (CBS) gene from M. grisea was used as an example of effects of transposon insertion on gene expression. Methods for isolating a cosmid, bacmid, or other DNA containing a gene of interest are known to those in the art.

Sif Transposition into a Cosmid: Transposition of Sif into the cosmid framework is carried out as described for the GPS-M mutagenesis system (New England Biolabs, Inc.). Briefly, 2 ul of the 10×GPS buffer, 70 ng of supercoiled pSIF, 8-12 ug of target cosmid DNA were mixed and taken to a final volume of 20 ul with water. 1 ul of transposase (TnsABC) was added to the reaction and incubated for 10 minutes at 37° C. to allow the assembly reaction to happen. After the assembly reaction 1 ul of start solution was added to the tube, mixed well and incubated for 1 hour at 37° C. followed by heat inactivation of the proteins at 75° C. for 10 min. Destruction of the remaining untransposed pSif was done by PIScel digestion at 37° C. for 2 hours followed by 10 min incubation at 75° C. to inactivate the proteins. Transformation of Top10F′ electrocompetent cells (Invitrogen) was done according to manufacturers recommendations. Sif-containing cosmid transformants were selected by growth on LB agar plates containing 50 ug/ml of hygromycin B (Sigma Chem. Co.) and 100 ug/ml of Ampicillin (Sigma Chem. Co.).

### EXAMPLE 3

### Analysis of Transposon Insertion

E. coli strains containing cosmids with transposon insertions were picked to 96 well growth blocks (Beckman Co.) containing 1.5 ml of TB (Terrific Broth, Sambrook et al. (1989) Molecular Cloning, a Laboratory Manual, Cold Spring Harbor Laboratory Press) supplemented with 50 ug/ml of ampicillin. Blocks were incubated with shaking at 37 C overnight. E. coli cells were pelleted by centrifugation and cosmids were isolated by a modified alkaline lysis method (Marra et al (1997) Genome Res 7: 1072-84). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

DNA sequences adjacent to the site of the insertion were collected and used to search DNA and protein databases using the BLAST algorithms (Altshul et. al. (1997) Nucleic Acids Res. 25:3389-3402). Three different insertions of SIF into the Magnaporthe grisea CBS gene were chosen for further analysis.

### EXAMPLE 4

### Preparation of Cosmid DNA and Transformation of the Fungus Magnaporthe grisea

Sif was used to perform targeted insertion on the CBS gene isolated from M. grisea. Three TAG-KO constructs (see WO 00/55346, PCT/US00/07317, U.S. Ser. No. 09/658,859) were used to transform M. grisea strain Guy11 (wild type) to generate mutant forms of CBS with different mutant alleles by homologous recombination in the native gene.

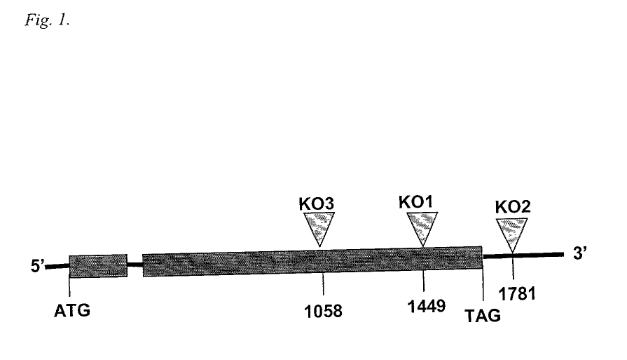

Cosmid DNA from the CBS transposon tagged cosmid clones was prepared using QIAGEN Plasmid Maxi Kit (QIAGEN), and digested by PI-PspI (New England Biolabs, Inc.). Fungal electro-transformation was performed essentially as described (Wu et al. (1997) MPMI. 10: 700-708). Briefly, M. grisea strain Guy 11 was grown in complete liquid media (Talbot et al. (1993) Plant Cell 5: 1575-1590 (PMID: 8312740)) shaking at 120 rpm for 3 days at 25° C. in the dark. Mycelia were harvested and washed with sterile H₂O and digested with 4 mg/ml beta-glucanase (InterSpex) for 4-6 hours to generate protoplasts. Protoplasts were collected by centrifugation and resuspended in 20% sucrose at the concentration of 2×10⁸ protoplasts/ml. 50 ul protoplast suspension was mixed with 10-20 ug of the cosmid DNA and pulsed using Gene Pulser II (BioRad) set with the following parameters: resistance 200 ohm, capacitance 25 uF, voltage 0.6 kV. Transformed protoplasts were regenerated in complete agar media (CM, Talbot et al. (1993) Plant Cell 5: 1575-1590 (PMID: 8312740)) with the addition of 20% sucrose for one day, then overlayed with CM agar media containing hygromycin B (250 ug/ml) to select transformants. Transformants were screened for homologous recombination events in the target gene by PCR (Hamer et al. (2001) Proc Natl Acad Sci USA 98: 5110-15). The positions of transposon insertion in the CBS TAG-KO mutants (KO1, K02, and K03) were shown in FIG. 1. In KO1 and KO3 CBS was interrupted in the coding region while KO2 carried the transposon insertion downstream of the stop codon (TAG).

### EXAMPLE 5

### Expression Analysis of CBS Mutants

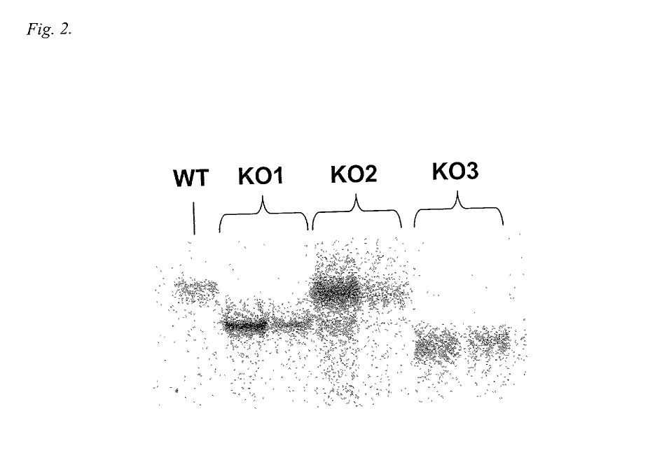

Total RNA was extracted from 3-day-old cultures of the wild type strain (Guy11) and CBS mutants grown for 3 days in Complete Media at 25 degrees Celsius in the dark. Expression of the CBS gene was analyzed by Northern hybridization (Sambrook et al.) using a fragment of the CBS gene as a probe. FIG. 2 shows that the gene was expressed in the wild type (WT) and in all the TAG-KO mutants. The CBS transcripts from KO1 and KO3 were considerably shorter than the wild type.

### EXAMPLE 6

### 3′-end Analysis of CBS Mutants

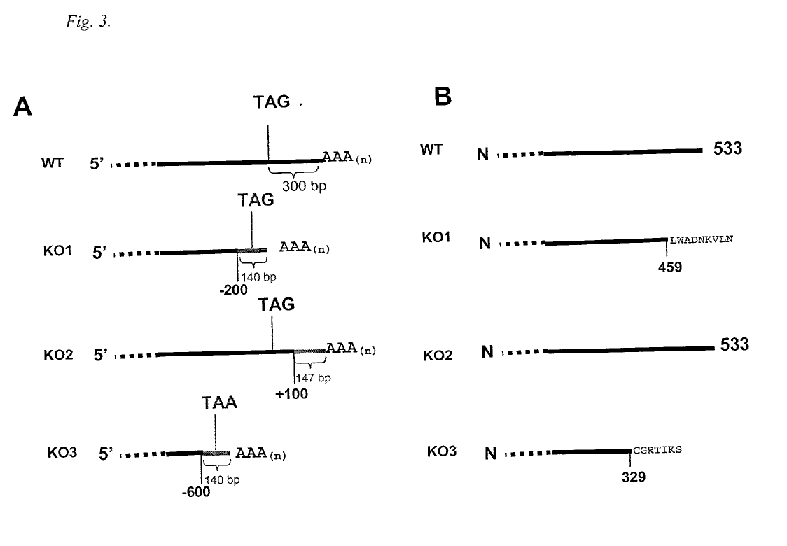

3′-end sequences of cDNA prepared from the different fungal strains were determined. cDNAs were prepared from RNA samples by reverse transcription-PCR. The 3′-ends of the CBS cDNAs were amplified by PCR with a CBS gene-specific primer and an oligo(dT) primer. Results showed that different truncated CBS-Sif transcripts were produced by the KO mutants (FIG. 3). They all contain some Tn7 transposon end sequences at the 3′ ends. Polyadenylation signals were found in those regions (SEQ ID NO: 1, 2, and 3). In the cases of KO1 and KO3, new stop codons (TAG or TAA) for the CBS gene could also be identified (FIG. 3A). KO2, on the other hand, retained all the wild type coding sequence for the protein.

The deduced CBS protein from the WT contains 533 amino acid (aa) residues (FIG. 3B). For KO1, the deduced CBS protein contained the first 459 aa of the WT sequence with an addition of 9 new aa as a result of the introduction of transposon sequences to the mRNA. For KO2, the CBS protein is expected to be the same as the WT protein. KO3 would produce the shortest CBS protein among the strains tested with the first 329 aa of the WT protein plus 7 novel aa at the C terminal.

### EXAMPLE 7

### Phenotypic Analysis of CBS Mutants

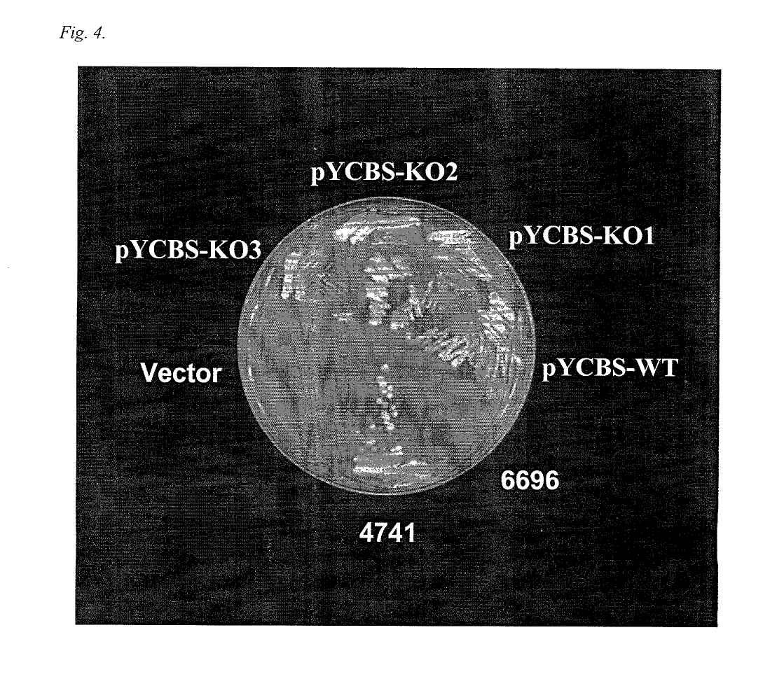

The different CBS cDNAs were cloned into pYes2. 1 vectors (Invitrogen) to make expression constructs. The constructs were transformed into a S. cerevisiae CBS mutant (Strain # 6696 from Research Genetics, Inc.) for functional analysis. S. cerevisiae requires CBS activities for the synthesis of cysteine. The CBS null mutant (6696) is a cysteine auxotroph. FIG. 4 shows the results for the yeast complementation experiments. WT, KO1, and KO2 CBS expression restored normal growth of yeast CBS mutants on minimal media without cysteine, indicating that their gene products are functional. Yeast CBS mutants transformed with KO3 constructs showed slower growth compared to other transformants, suggesting that the KO3 gene product was only partially active. Thus, the catalytic domain of CBS appears to be still intact in KO3, but some positive regulatory components might have been removed as a result of the transposon insertion.

These results demonstrated that Sif insertion results in termination of the transcription of the CBS gene in M. grisea. It is a novel discovery that Tn7, a prokaryotic transposon carries 3′ end formation site information unique to eukaryotic genes. The truncated messages were chimeric and polyadenylated.

### EXAMPLE 8

### Overexpression and Purification of CBS Protein

The CBS protein is produced as described in Jhee et al. (2000) J Biol Chem 275: 11541-4 (PMID: 10766767): A 1-liter culture of E. coli XL1-blue are transformed with a vector for the overexpression of CBS, such as pSEC (see Jhee et al. (2000) J Biol Chem 275:11541-4 (PMID: 10766767)) and grown at 37° C. in Super Broth (BioWhittaker or KD Medical) containing tryptone (12 g/liter), yeast extract (24 g/liter), glycerol (6.3 g/liter), K₂HPO₄ (12.5 g/liter), KH₂PO₄ (3.8 g/liter),—aminolevulinic acid (50 mg/liter), ampicillin (100 mg/liter), and 20 ml of 50-fold concentrated Vogel and Bonner minimal medium (Vogel and Bonner (1956) J Biol Chem 218: 97-106). A 10% inoculum is added to the medium, and growth proceeds for ˜3-4 h until the OD650 reachs 2.5. IPTG is added to 0.1 mM, and growth is continued at 30° C. for 14-18 h. Cells are harvested by centrifugation, washed with 0.85% NaCl containing 1 mM dithiothreitol, resuspended in Buffer BP (50 mM sodium/bicine, pH 7.8, containing 10 mM EDTA, 10 mM -mercaptoethanol, 0.1 mM PLP, 1 mM PMSF, 0.1 mM TLCK, 0.1 mM TPCK, 1 mg/liter aprotinin, 2 mg/liter leupeptin, 2 mg/liter pepstatin, and 1 mM benzamidine-HCl), and disrupted by passaging twice through a French press at 8,000 p.s.i. The suspension is centrifuged at 12,000×g for 30 min.

Eight ml of a 2% solution of protamine sulfate in Buffer BP is added dropwise to the crude extract with stirring at room temperature followed by additional stirring for 20 min. The suspension is centrifuged, and the precipitate discarded. The supernatant solution is fractionated with ammonium sulfate at pH 7.5. The 30-60% ammonium sulfate fraction is dialyzed against three changes of Buffer BP at 4° C. for 6 h. The dialyzed enzyme solution is loaded onto a 2.5×20-cm column of DEAE-Sephacel, which is then washed with 300 ml of Buffer BP. The enzyme is eluted with a 1-liter linear gradient from 0 to 0.5 M NaCl in Buffer BP. Fractions are analyzed by SDS-polyacrylamide gel electrophoresis. The relevant fractions are pooled, concentrated, and dialyzed against Buffer KP (10 mM potassium phosphate, pH 7.8, containing 10 mM EDTA, 10 mM -mercaptoethanol, 0.1 mM PLP, 1 mM PMSF, 0.1 mM TLCK, 0.1 mM TPCK, 1 ml/liter aprotinin, 2 mg/liter leupeptin, 2 mg/liter pepstatin, and 1 mM benzamidine-HCl at pH 7.8).

The dialyzed enzyme solution is applied to a Gigapite column (3.1×27 cm) equilibrated with Buffer KP. Gigapite is a modified form of hydroxyapatite that has large particles and gives a high flow rate. The column is washed with 400 ml of Buffer KP followed by 300 ml of Buffer KP that contains 50 mM potassium phosphate. The enzyme is eluted with a 1.8-liter linear gradient ranging from 50 to 400 mM potassium phosphate in Buffer KP. The relevant fractions are concentrated to 10-30 mg/ml, dialyzed against Buffer K (50 mM potassium phosphate, pH 7.5, containing 1 mM EDTA, 1 mM dithiothreitol, and 0.02 mM PLP), and stored at 85° C. All procedures are completed within 72 h to limit proteolysis.

### EXAMPLE 9

### In vitro Binding Assay

Procedures for assaying binding of the CBS protein and/or mutant forms thereof, such as to the Huntington protein, in vitro and by means of the yeast two-hybrid system may be found in Boutell et al. (1998) Hum Mol Genet 7: 371-8 (PMID: 9466992).

### EXAMPLE 10

### Transcriptional Assay

Assays for assessing the effects of CBS or mutant forms thereof on transcription may be found in Hansen and Johannesen (2000) Mol Gen Genet 263: 535-42 (PMID: 10821189) and Outinen et al. (1998) Biochem J 332: 213-21 (PMID: 9576870).

### EXAMPLE 11

### Biochemical Assay

Cystathione beta synthase (CBS) catalyzes the conversion of homocysteine to cystathionine, and enzymatic activity of CBS and/or mutant forms thereof can be biochemically assayed as may be described in Jhee et al. (2000) J Biol Chem. 275: 11541-4 (PMID: 10766767): Protein concentrations are determined by the Coomassie Blue protein assay reagent (Pierce) using bovine serum albumin as a standard or from the specific absorbance of purified CBS at 280 nm. CBS activity is determined by a modification of a standard method (Kraus et al. (1978) J Biol Chem 253: 6523-6528 (PMID: 681363)). The reaction mixture, which contains 200 mM Tris-HCl, pH 8.6, 20 μM PLP, 0.25 mg/ml bovine serum albumin, 5 mM L-[U-¹⁴C]serine (800 cpm/nmol), and CBS (0.02-0.1 μg) in 18 μl, is preincubated for 5 min at 37° C. The reaction is initiated by adding 2 μl of 50 mM homocysteine to 5 mM and is terminated after 10-15 min by adding 5 μl of 50% trichloroacetic acid. After the mixture was centrifuged for 3 min, 5 μl of the supernatant is applied to a cellulose thin layer chromatography plate (Kodak). The product, L-[¹⁴C]cystathionine, is separated from L-[¹⁴C]serine by ascending thin layer chromatography in 2-propanol/formic acid/H20 (80/6/20 v/v). Radioactivity of the product is determined by Phosphorimager (Molecular Dynamics). One unit of activity is defined as the production of 1 μmol of L-cystathionine/h at 37° C.

While the foregoing describes certain embodiments of the invention, it will be understood by those skilled in the art that variations and modifications may be made and still fall within the scope of the invention.

What is claimed is:

1. A method for preparing a functionally distinct mutant form of a gene and/or a mutant gene product comprising:
a) inserting at least one Tn7 transposon in vitro into a gene to produce a mutant form of said gene; and
b) assaying said mutant gene and/or its mutant gene product for a change in activity or function compared to said activity or function for the gene and/or its gene product;
wherein a change indicates the mutant form and/or its mutant gene product is functionally distinct from said gene and/or its gene product.

2. The method of claim 1, wherein said Tn7 transposon is a Sif transposon.

3. The method of claim 1, wherein said gene is from a filamentous fungus.

4. The method of claim 1, wherein said gene is selected from the group consisting of Magnaporthe grisea and Mycosphaerella graminicola.

5. The method of claim 1, wherein said assaying is performed using an in vitro binding assay.

6. The method of claim 1, wherein said assaying is performed using an enzymatic assay.

7. The method of claim 1, wherein said assaying is performed using a transcriptional assay.

8. A method for determining a functional domain of a gene and/or its gene product comprising:
a) inserting at least one Tn7 transposon in vitro into a gene to produce a mutant form of said gene;
b) assaying said mutant gene and/or its mutant gene product for a change in activity or function compared to said activity or function for the gene and/or its gene product; and
c) determining the sequence alteration in at least one mutant form of a gene and/or a mutant gene product as compared to the sequence of the gene and/or its gene product as a result of said insertion;
wherein an alteration in sequence indicates that said sequence alteration corresponds to at least part of a functional domain of the gene and/or its gene product.

9. The method of claim 8, wherein said Tn7 transposon is a Sif transposon.

10. The method of claim 8, wherein said gene is from a filamentous fungus.

11. The method of claim 8, wherein said gene is selected from the group consisting of Magnaporthe grisea and Mycosphaerella graminicola.

12. The method of claim 8, wherein said assaying is performed using an in vitro binding assay.

13. The method of claim 8, wherein said assaying is performed using an enzymatic assay.

14. The method of claim 8, wherein said assaying is performed using a transcriptional assay.

15. A method for preparing a functionally distinct mutant form of a gene and/or a mutant gene product comprising:
a) inserting at least one Tn7 transposon in vitro into a gene to produce a mutant form of said gene;
b) transforming at least one host with said mutant form to give a transformant; and
c) assaying:
1) said mutant gene and/or its mutant gene product for a change in activity or function compared to said activity or function for the gene and/or its gene product, and/or
2) said transformant for a change in phenotype;
wherein a change detected in c1) and/or c2) indicates the mutant form and/or its mutant gene product is a functionally distinct mutant form of said gene and/or its gene product.

16. The method of claim 15, wherein said host is a filamentous fungus.

17. The method of claim 15, wherein said filamentous fungus is selected from the group consisting of Magnaporthe grisea and Mycosphaerella graminicola.

18. The method of claim 15, wherein said gene is from a filamentous fungus.

19. The method of claim 15, wherein said gene is selected from the group consisting of Magnaporthe grisea and Mycosphaerella gram inicola.

20. The method of claim 15, wherein said assaying is performed using a phenotypic assay.

21. The method of claim 15, wherein said assaying is performed using a yeast complementation assay.

22. The method of claim 15, wherein said assaying is performed using a yeast interaction trap assay.

23. The method of claim 15, wherein said assaying is performed using an enzymatic assay.

24. The method of claim 15, wherein said assaying is performed using a transcriptional assay.

25. The method of claim 15, wherein said Tn7 transposon is a Sif transposon.

26. A method for preparing a functionally distinct mutant form of a gene and/or a mutant gene product comprising:
a) inserting at least one Tn7 transposon in vitro into a gene to produce a mutant form of said gene;
b) transforming at least one host with said mutant form to give a transformant;
c) assaying:
1) said mutant gene and/or its mutant gene product for a change in activity or function compared to said activity or function for the gene and/or its gene product, and/or
2) said transformant for a change in phenotype;
d) determining the sequence alteration in at least one mutant form of a gene and/or a mutant gene product as compared to the sequence of the gene and/or its gene product as a result of said insertion;
wherein an alteration in sequence indicates that said sequence alteration corresponds to at least part of a functional domain of the gene and/or its gene product.

27. The method of claim 26, wherein said host is a filamentous fungus.

28. The method of claim 26, wherein said filamentous fungus is selected from the group consisting of Magnaporthe grisea and Mycosphaerella graminicola.

29. The method of claim 26, wherein said gene is from a filamentous fungus.

30. The method of claim 26, wherein said gene is selected from the group consisting of Magnaporthe grisea and Mycosphaerella graminicola.

31. The method of claim 26, wherein said assaying is performed using a phenotypic assay.

32. The method of claim 26, wherein said assaying is performed using a yeast complementation assay.

33. The method of claim 26, wherein said assaying is performed using a yeast interaction trap assay.

34. The method of claim 26, wherein said assaying is performed using an enzymatic assay.

35. The method of claim 26, wherein said assaying is performed using a transcriptional assay.

36. The method of claim 26, wherein said Tn7 transposon is a Sif transposon.


SEQUENCE LISTING (3 sequences)

SEQ ID NO: 1 - DNA, 282 bp, Magnaporthe grisea
  polyA_signal (171)..(175)
gggttggttacacttggaaacatgctgagctacgtttctcgcggccgcgtcaagaccaatagccctgtcagcgacgtcatgtttgactttggccgacttgacgaggttatcaccgatctgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaaaaaaaaaaaaaaaaaaaaaaa

SEQ ID NO: 2 - DNA, 260 bp, Magnaporthe grisea
  misc_feature (248)..(260): polyA tail
aagctctgaggttttgagagaccttgccaatgtgggtgcagccggtgttttgacttgatcttcttaggtgttttttatgttattttcttacccgtccttttgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactaaaaaaaaaaaaa

SEQ ID NO: 3 - DNA, 237 bp, Magnaporthe grisea
  polyA_signal (89)..(93)
acctgggcgaggacgacgttgtggttgtcgtcctcccggatagcatccgcagctacctctccaagtttgccgacgattgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaaaaaaaaaaaaaaaaaaa